(19) United States
(12) Patent Application Publication
(10) Pub. No.: US 2019/0133956 A1
(43) Pub. Date: May 9, 2019

(54) REINFORCED PHARMACEUTICAL DOSAGE FORM

(71) Applicant: GRÜNENTHAL GMBH, Aachen (DE)

(72) Inventors: Marcel HAUPTS, Stolberg (DE); Klaus WENING, Köln (DE); Carmen STOMBERG, Korschenbroich (DE); Siegfried EBNER, Roetgen (DE)

(73) Assignee: GRÜNENTHAL GMBH, Aachen (DE)

(21) Appl. No.: 16/240,943

(22) Filed: Jan. 7, 2019

Related U.S. Application Data
(63) Continuation of application No. PCT/EP2017/066907, filed on Jul. 6, 2017.

(30) Foreign Application Priority Data
Jul. 6, 2016 (EP) 16178238.8

(51) Int. Cl. A61K 9/20

(52) U.S. Cl. CPC A61K 9/2031; A61K 9/2072; A61K 9/16; A61K 9/2095; A61K 9/2054

(57) ABSTRACT

The invention relates to a reinforced pharmaceutical dosage form comprising a pharmacologically active ingredient and fibers. The reinforced pharmaceutical dosage form is tamper-resistant and thus useful for the avoidance of drug abuse or misuse. The invention also relates to the preparation of such dosage forms and their use in therapy.

This application is a continuation application of PCT/EP2017/066907, filed Jul. 6, 2017, which claims priority of European Patent Application No. 16178238.8, filed on Jul. 6, 2016, the entire contents of which are incorporated herein by reference,

### FIELD OF THE INVENTION

The invention relates to a reinforced pharmaceutical dosage form comprising a pharmacologically active ingredient and fibers. The reinforced pharmaceutical dosage form is tamper-resistant and thus useful for the avoidance of drug abuse or misuse. The invention also relates to the preparation of such dosage forms and their use in therapy.

### BACKGROUND OF THE INVENTION

A large number of drugs have a potential for being abused or misused, i.e. they can be used to produce effects which are not consistent with their intended use. Thus, e.g. opioids which exhibit an excellent efficacy in controlling severe to extremely severe pain are frequently abused to induce euphoric states similar to being intoxicated. In particular, active substances which have a psychotropic effect are abused accordingly.

To enable abuse, the corresponding pharmaceutical dosage forms, such as pharmaceutical dosage forms or capsules are crushed, for example ground by the abuser, the drug is extracted from the thus obtained powder using a preferably aqueous liquid and after being optionally filtered through cotton wool or cellulose wadding, the resultant solution is administered parenterally, in particular intravenously. This type of dosage results in an even faster diffusion of the active substance compared to the oral abuse, with the result desired by the abuser, namely the kick. This kick or these intoxication-like, euphoric states are also reached if the powdered pharmaceutical dosage form is administered nasally, i.e. is sniffed.

Various concepts for the avoidance of drug abuse have been developed.

It has been proposed to incorporate in pharmaceutical dosage forms aversive agents and/or antagonists in a manner so that they only produce their aversive and/or antagonizing effects when the pharmaceutical dosage forms are tampered with. However, the presence of such aversive agents is principally not desirable and there is a need to provide sufficient tamper-resistance without relying on aversive agents and/or antagonists.

Another concept to prevent abuse relies on the mechanical properties of the pharmaceutical dosage forms, particularly an increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, the pulverization, necessary for abuse, of the pharmaceutical dosage forms by the means usually available to a potential abuser is prevented or at least complicated.

The mechanical properties, particularly the high breaking strength of these pharmaceutical dosage forms renders them tamper-resistant. In the context of such tamper-resistant pharmaceutical dosage forms it can be referred to, e.g., WO 2005/016313, WO 2005/016314, WO 2005/063214, WO 2005/102286, WO 2006/002883, WO 2006/002884, WO 2006/002886, WO 2006/082097, WO 2006/082099, and WO2009/092601.

US 2015/0283086 relates to a process for the production of an abuse-proofed dosage form. The dosage form may have spatially separated subunits, wherein one subunit forms a core which is enclosed by another subunit, wherein the latter comprises at least one channel which leads from the core to the surface of the dosage form.

One further approach to render pharmaceutical dosage forms tamper resistant or abuse resistant is to include particles in the dosage forms, wherein the particles comprise the pharmacologically active ingredient and have an increased breaking strength and/or resistance to dissolution.

US 2013/0280338 refers to a tamper-resistant pharmaceutical dosage form comprising a pharmacologically active ingredient embedded in a prolonged release matrix, which provides prolonged release of the pharmacologically active ingredient, resistance against solvent extraction, resistance against grinding, and resistance against dose-dumping in aqueous ethanol. The dosage form may comprise particulates which may have a spherical shape and an aspect ratio of at most 1.4.

US 2013/0330409 discloses a tamper resistant dosage form, comprising non-stretched melt extruded particulates comprising a drug and a matrix; wherein said melt extruded particulates are present as a discontinuous phase in said matrix. The particulates may have a significantly smaller diameter than conventional particulates. The particulates may have a diameter in the range of 100 μm to 900 μm and a length in the range of 200 to 1000 μm.

WO 2012/061779 refers to abuse-deterrent drug formulations comprising a plurality of discrete domains uniformly dispersed in a pharmaceutically acceptable matrix, wherein said domains have high fracture toughness and comprise at least one polymer and at least one abuse-relevant drug. The domains have an average size of about 100 μm to about 1000 μm. The domains may be composed of a filler and/or a fiber, the latter may be a cellulosic excipient.

US 2005/0136112 relates to an oral medicament delivery system comprising a pharmaceutical composition comprising a flexible matrix, said matrix formed of a plurality of fibers comprising a collagen-based carrier and a medicament, the composition orally dissolvable to deliver a unit dose of the medicament to a patient. The delivery system does not resemble a pill or tablet but has a fibrous appearance and structure, microscopically, which renders the composition mucous membrane adhesive, flexible and orally dissolvable. Furthermore, this dose delivery form can be torn, cut or severed with scissors to produce smaller dosage forms.

Besides tampering of pharmaceutical dosage forms in order to abuse the drugs contained therein, the potential impact of concomitant intake of ethanol on the in vivo release of drugs from modified release oral formulations (dose-dumping) has recently become an increasing concern. Controlled or modified release formulations typically contain a higher amount of the pharmacologically active ingredient relative to its immediate release counterpart. If the controlled release portion of the formulation is easily defeated, the end result is a potential increase in exposure to the active drug and possible safety concerns. In order to improve safety and circumvent intentional tampering (e.g. dissolving a controlled release pharmaceutical dosage form in ethanol to extract the drug), a reduction in the dissolution of the modified release fractions of such formulations, in ethanol, may be of benefit. Accordingly, the need exists to develop new formulations having reduced potential for dose dumping in alcohol.

The properties of the pharmaceutical dosage forms of the prior art, however, are not satisfactory in every respect.

It is an object of the invention to provide pharmaceutical dosage forms which have advantages compared to the pharmaceutical dosage forms of the prior art. The pharmaceutical dosage forms should preferably provide improved tamper resistance, especially against mechanical disruption, such as hammering, grinding, crushing and cutting. Furthermore, the pharmaceutical dosage forms should preferably provide improved resistance against solvent extraction also in non-aqueous solvents.

This object has been achieved by the subject-matter of the patent claims.

It has been surprisingly found that pharmaceutical dosage forms can be reinforced thereby substantially improving the abuse deterrent properties of the pharmaceutical dosage forms, particularly with respect to mechanical disruption such as cutting.

Further, it has been surprisingly found that reinforced pharmaceutical dosage forms can be manufactured by three-dimensional printing technology, particularly fused deposition modeling. This technology allows both, printing a filament material comprising (microscopic) fibers in a polymer matrix or printing a filament material to form the (macroscopic) fibers as such.

Still further, it has been surprisingly found that it is possible to manufacture tamper-resistant pharmaceutical dosage forms by three-dimensionally printing of polymer types that are not erodible under physiological conditions and hence have not been conventionally used for the manufacture of tamper-resistant pharmaceutical dosage forms. As three-dimensional printing technology allows for producing microstructures that allow the release medium, e.g. gastric juice, to enter the dosage form in a controlled and predetermined manner, non-erodible polymers can be used that are highly resistant against various chemicals, e.g. polyether ether ketone (PEEK). After the pharmacologically active ingredients have been released from the pharmaceutical dosage forms through said microstructures, the remainder is excreted by the gastrointestinal tract.

A first aspect of the invention relates to a reinforced pharmaceutical dosage form comprising a pharmacologically active ingredient and fibers.

The pharmaceutical dosage form comprises fibers which may either be contained in a polymer matrix (composite material) and/or which may comprise or essentially consist of a polymer or a polymer matrix themselves.

When the fibers are contained in a polymer matrix (composite material), the fibers are preferably of microscopic size and made of a material that differs from the material of the polymer matrix, i.e. from the polymers of the polymer matrix.

When the fibers comprise or essentially consist of a polymer or a polymer matrix themselves, the fibers are preferably of macroscopic size and as such are arranged to form a structural element of the dosage form, e.g. a layer of a multitude of fibers that are arranged in parallel to one another and that are in contact with one another, or a long fiber that is arranged in a serpentine-like manner such that sections of said long fiber are arranged in parallel to one another and that are in contact with one another.

Such arrangement in a serpentine-like manner preferably results in a reinforced layer of the pharmaceutical dosage form wherein macroscopic fibers (strands of material) are arranged in parallel to one another and are in contact with one another thereby providing this individual reinforced layer with anisotropic mechanical properties. Preferably, several of such reinforced layers are arranged above one another wherein the macroscopic fibers in each reinforced layer are parallel to one another and thus the mechanical properties are anisotropic with respect to the direction of orientation of fibers. Preferably, adjacent reinforced layers that are arranged above one another have different orientations of fibers such that the resultant anisotropic mechanical properties are differently orientated as well. The reinforced layers may, for example, be printed by melt extruding a polymer composition through a micro nozzle such that the congealing material forms fibers. Alternatively or additionally, said polymer composition may comprise fibers. The pharmacologically active ingredient may be contained in such a reinforced layer or may be contained in a separate layer that is positioned between two or more such reinforced layers.

For the purpose of the specification, a fiber is preferably regarded as a filament, typically a slender and greatly elongated natural or synthetic filament. Preferably, a fiber is regarded as a thread or a structure or object resembling a thread.

The pharmaceutical dosage form according to the invention is particularly useful for pharmacologically active ingredients with abuse potential, as the pharmaceutical dosage form is characterized by a specific mechanical strength and/or resistance against chemicals and solvents.

The specific mechanical strength can be achieved by arranging fibers in a layered structure, wherein the fibers may be oriented or arbitrarily arranged. Due to the mechanical properties of the material that is used to prepare such layers (e.g. fiber reinforced polymer matrix or macroscopic polymer fibers as such) every layer already exhibits improved mechanical strength. For example, when the layers comprise a hard material, the exhibit improved resistance against cutting in a direction orthogonal to the direction of orientation of the fibers.

Preferably, several layers are arranged above one another with different orientation such that the pharmaceutical dosage form as such has improved mechanical strength. Even if a potential abuser is able to cut a certain layer e.g. with a knife, in a particular direction, one of the layers below said (cut) certain layer will exhibit improved resistance against cutting due to its specific orientation of the fibers so that cutting the entire dosage form is prevented or at least substantially impeded.

Alternatively or additionally, the pharmaceutical dosage form may comprise the fibers in form of a preformed web or fabric, e.g. bags, jackets, sleeves or tubes, which are made of a material exhibiting outstanding mechanical strength, e.g. aramid.

Resistance against chemicals or solvents may be achieved by means of chemical inert polymers such as polyether ether ketone (PEEK). As this material is not erodible in body fluids, the pharmaceutical dosage form is provide with pockets that may act as canals for the release medium thereby allowing the release medium to enter the inside of the dosage form and to extract the pharmacologically active ingredient in a defined and controlled manner.

In a preferred embodiment of the pharmaceutical dosage form according to the invention, the fibers are distributed over the whole pharmaceutical dosage form, preferably homogeneously.

In another preferred embodiment of the pharmaceutical dosage form according to the invention, the fibers are locally concentrated in distinct sections of the pharmaceutical dosage form, which preferably have a size of at least 0.2 mm³, such that the pharmaceutical dosage form comprises
(i) first sections comprising fibers and second sections not comprising fibers; and/or
(ii) first sections comprising a first quantity of fibers per volume element and second sections comprising a second quantity of fibers per volume element, wherein said first quantity and said second quantity differ from one another.

In a preferred embodiment, the pharmaceutical dosage form according to the invention comprises an inner core (first section) which contains the pharmacologically active ingredient or a major amount thereof, but no fibers or only a minor amount thereof; and an outer sphere (second section) surrounding said inner core, which outer sphere contains the fibers or a major amount thereof, but no pharmacologically active ingredient or only a minor amount thereof.

In another preferred embodiment, the pharmaceutical dosage form according to the invention comprises an inner core (first section) which contains the pharmacologically active ingredient or a major amount thereof as well as the fibers or a major amount thereof; and an outer sphere (second section) surrounding said inner core, which outer sphere contains no fibers or only a minor amount thereof, and no pharmacologically active ingredient or only a minor amount thereof.

In a preferred embodiment, at least a portion of the fibers is oriented in essentially the same direction.

Orientation may be in two-dimensional orientations or three-dimensional orientation.

In a preferred embodiment, at least a portion of the fibers is non-oriented i.e. arranged arbitrarily such that the fibers do not have a common direction of orientation (see FIGS. 1 and 2).

In another preferred embodiment, the pharmaceutical dosage form according to the invention is two-dimensionally fiber reinforced and comprises a laminated structure in which the fibers are only aligned along the plane in x-direction and y-direction of the material. This means that essentially no fibers are aligned in the z-direction (see FIG. 3).

In another preferred embodiment, the pharmaceutical dosage form according to the invention is three-dimensionally fiber reinforced incorporating fibers are aligned in the x-direction, y-direction and z-direction. This may be achieved, e.g. by arranging the fibers in a coiled arrangement, e.g. like in a wool coil or wool pouf.

In a preferred embodiment, the fibers are of macroscopic size. Preferably, the fibers comprise or essentially consist of one or more polymers. Preferably, the fibers are arranged essentially in parallel to one another and preferably in contact with one another thereby forming a plane which is preferably a layer of the pharmaceutical dosage form according to the invention (see FIG. 4). Said plane may be formed by separate fibers or by a long fiber that is arranged in a serpentine-like manner. Patterns of this and similar type can be easily prepared by three-dimensional printing technology.

In a preferred embodiment, the pharmaceutical dosage form according to the invention comprises at least one layer (3) comprising or essentially consisting of fibers which are oriented in different directions of orientation, wherein said different directions of orientation lie essentially within the plane of said layer (see FIG. 2).

In another preferred embodiment, the pharmaceutical dosage form according to the invention comprises a plurality of layers, preferably, 3, 4, 5, 6, 7, 8, 9, or 10 layers, wherein each layer comprises fibers which are oriented in essentially a same direction of orientation, wherein the direction of orientation of adjacent layers differs from one another (see FIGS. 5 and 6).

Preferably, the direction of orientation of all layers differs from one another.

Preferably, the angle of the two different directions of orientation of two adjacent layers, preferably of all different direction of orientation of all layers relative to one another, is at least 1° (0.0175 rad), more preferably at least 20, still more preferably at least 30, yet more preferably at least 40, even more preferably at least 50, most preferably at least 60, and in particular at least 7°.

Preferably, the angle of the two different directions of orientation of two adjacent layers is a function of the number of layers. When the pharmaceutical dosage form has n layers comprising fibers which are oriented in n different directions of orientation, the angle of the two different directions of orientation of two adjacent layers is preferably (180°/n)±10°, more preferably (180°/n)±9°, still more preferably (180°/n)±8°, yet more preferably (180°/n)±7°, even more preferably (180°/n)±6°, most preferably (180°/n)±5°, and in particular (180°/n)±4°.

Preferably, the direction of orientation of each layer lies essentially within the plane of said layer (see FIGS. 5 and 6).

It has been surprisingly found that multilayered pharmaceutical dosage forms of this type, i.e. with various directions of orientation in various layers, provides resistance against cutting.

In a preferred embodiment, the pharmaceutical dosage form according to the invention comprises a woven or nonwoven fabric comprising the fibers. Thus, the fibers may be present in form of sheets or mats. The four major ways to manufacture such sheets or mats is through the textile processing techniques of weaving, knitting, braiding and stitching.

In a particularly preferred embodiment, the fibers of the woven or nonwoven fabric comprise or essentially consist of a non-erodible polymer, more preferably of a polyamide, still more preferably of aramid.

In a preferred embodiment, the woven or nonwoven fabric is provided in form of pre-prepared little bags, jackets, sleeves or tubes of appropriate size in which tablet cores are placed in the course of manufacture of the pharmaceutical dosage forms. Preferably, the size of said little bags, jackets, sleeves or tubes is precisely adjusted to the size of said cores such that both fit and adapt to one another in a compact and close manner.

Preferably, in the course of manufacture of such pharmaceutical dosage forms, excipients are deposited at the outer surface of the thus arranged little bags, jackets, sleeves or tubes such that they are not visible from the outside (see FIG. 7). Deposition of suitable excipients in suitable amounts can be achieved in a known manner, e.g. by spraying, dipping or any other coating techniques, by three-dimensional-printing, or hot-melt extrusion.

Preferably, the woven or nonwoven fabric comprises pores that are permeable for the release medium, e.g. gastric juice, such that release of the pharmacologically active ingredient which is preferably contained in the core, i.e. in the inside of said little bags, jackets, sleeves or tubes can easily proceed through said pores.

Another aspect of the invention relates to a pharmaceutical dosage form that may be reinforced by fibers, but that does not necessarily have to be reinforced by fibers. In a preferred embodiment, said pharmaceutical dosage form does not comprise any fibers within the meaning of the present invention.

According to this aspect, the invention relates to a pharmaceutical dosage form comprising a pharmacologically active ingredient and a polymer matrix, wherein the polymer matrix is not erodible under physiological conditions, and wherein the pharmaceutical dosage form comprises one or more pockets that serve as canals allowing the release medium to penetrate from the outside through the pockets into the pharmaceutical dosage form.

According to a preferred embodiment, the pockets have openings at the outer surface of the pharmaceutical dosage form such that upon contact with gastric or intestinal fluid, said fluid may penetrate the pockets and thus reach interior areas of the pharmaceutical dosage form (see FIG. 9).

According to another preferred embodiment, the pockets do not have openings at the outer surface of the pharmaceutical dosage form, whereas such openings are blocked with an erodible material. Thus, according to this preferred embodiment, the pockets are initially closed. Upon contact with gastric fluid, in the course of erosion and release of the pharmacologically active ingredient, however, the openings are set free such that subsequently the gastric or intestinal fluid may penetrate the pockets and thus reach interior areas of the pharmaceutical dosage form.

In either embodiment the pockets contribute to the overall release kinetics of the pharmacologically active ingredient from the pharmaceutical dosage form, as they shorten diffusion and erosion pathways of the gastric fluid.

Preferably, the one or more pockets have an average diameter of at least 100 μm, or of at least 110 μm, or of at least 120 μm, or of at least 130 μm, or of at least 140 μm, or of at least 150 μm, or of at least 160 μm, or of at least 170 μm, or of at least 180 μm, or of at least 190 μm, or of at least 200 μm, or of at least 210 μm, or of at least 220 μm, or of at least 230 μm, or of at least 240 μm, or of at least 250 μm, or of at least 260 μm, or of at least 270 μm, or of at least 280 μm, or of at least 290 μm, or of at least 300 μm, or of at least 350 μm, or of at least 400 μm, or of at least 450 μm, or of at least 500 μm, or of at least 550 μm, or of at least 600 μm, or of at least 650 μm, or of at least 700 μm, or of at least 750 μm, or of at least 800 μm or of at least 850 μm, or of at least 900 μm, or of at least 950 μm, or of at least 1000 μm, or of at least 1050 μm, or of at least 1100 μm, or of at least 1150 μm, or of at least 1200 μm, or of at least 1250 μm, or of at least 1300 μm, or of at least 1350 μm, or of at least 1400 μm, or of at least 1450 μm, or of at least 1500 μm.

Preferably, the one or more pockets have an average diameter of at most 1500 μm, or of at most 1400 μm, or of at most 1300 μm, or of at most 1200 μm, or of at most 1100 μm, or of at most 1000 μm, or of at most 900 μm, or of at most 990 μm, or of at most 980 μm, or of at most 970 μm, or of at most 960 μm, or of at most 950 μm, or of at most 940 μm, or of at most 930 μm, or of at most 920 μm, or of at most 910 μm.

Preferably, the dosage form comprises at least two pockets which have essentially the same diameter.

In another preferred embodiment, the dosage form comprises at least two pockets which have different diameters.

Preferably, the one or more pockets have an average length of at least 500 μm, or of at least 550 μm, or of at least 600 μm, or of at least 650 μm, or of at least 700 μm, or of at least 750 μm, or of at least 800, m or of at least 850 μm, or of at least 900 μm, or of at least 950 μm, or of at least 1000 μm, or of at least 1050 μm, or of at least 1100 μm, or of at least 1150 μm, or of at least 1200 μm, or of at least 1250 μm, or of at least 1300 μm, or of at least 1350 μm, or of at least 1400 μm, or of at least 1450 μm, or of at least 1500 μm, or of at least 1550 μm, or of at least 1600 μm, or of at least 1650 μm, or of at least 1700 μm, or of at least 1750 μm, or of at least 1800 μm, or of at least 1850 μm, or of at least 1900 μm, or of at least 2000 μm, or of at least 2050 μm, or of at least 2100 μm, or of at least 2150 μm, or of at least 2200 μm, or of at least 2250 μm, or of at least 2300 μm, or of at least 2350 μm, or of at least 2400 μm, or of at least 2450 μm, or of at least 2500 μm.

Preferably, the one or more pockets have an average length of at most 2500 μm, or of at most 2400 μm, or of at most 2300 μm, or of at most 2200 μm, or of at most 2100 μm, or of at most 2000 μm, or of at most 1900 μm, or of at most 1990 μm, or of at most 1980 μm, or of at most 1970 μm, or of at most 1960 μm, or of at most 1950 μm, or of at most 1940 μm, or of at most 1930 μm, or of at most 1920 μm, or of at most 1910 μm.

In a preferred embodiment, the dosage form has an outer shape that describes at least one circle and the length of the one or more pockets is at least half of the diameter of the circle.

In another preferred embodiment, the dosage form has an outer shape that describes at least one circle and the length of the one or more pockets is at least half of the radius of the circle.

In a preferred embodiment, the dosage form comprises at least two pockets which have essentially the same length.

In another preferred embodiment, the dosage form comprises at least two pockets which have different lengths.

Preferably, at least one of the one or more pockets has two openings which are at opposite sides of the dosage form.

Preferably, the dosage form comprises
at least two pockets, or at least 3, or at least 4, or at least 5, or at least 6, or at least 7, or at least 8, or at least 9, or at least 20, or at least 30 pockets; or
at most 10 pockets, or at most 9, or at most 8, or at most 7, or at most 6, or at most 5, or at most 4, or at most 3 pockets.

In a preferred embodiment, the dosage form comprises at least two pockets which are situated at opposite sides of the dosage form.

In another preferred embodiment, the dosage form comprises at least two pockets which are situated at the same side of the dosage form.

Preferably, the pharmaceutical dosage form according to the invention comprises a polymer matrix that is reinforced with the fibers.

In a preferred embodiment, the polymer matrix is erodible in gastric juice.

In another preferred embodiment, the polymer matrix is not erodible under physiological conditions, i.e. erodible neither in gastric juice nor in any other body fluid.

In a preferred embodiment, the polymer matrix comprises a thermoplastic polymer.

In another preferred embodiment, the polymer matrix comprises a cured polymer, e.g. a radiation cured polymer or a heat cured polymer (thermoset).

The polymer components of the polymer matrix are not particularly limited. Principally, the polymer matrix may comprise and polymer that has been approved for pharmaceutical purposes and that is compatible with the fibers or useful for the manufacture of fibers.

Preferably, the polymer matrix comprises a polymer selected from the group consisting of polyalkylene oxides (preferably polymethylene oxide, polyethylene oxide, polypropylene oxide), polyethylenes, polypropylenes, polyvinyl chlorides, polycarbonates, polystyrenes, polyacrylates, poly(hydroxy fatty acids), poly(hydroxyvaleric acids); polycaprolactones, polyvinyl alcohols, polyesteramides, polyethylene succinates, polylactones, polyglycolides, cellulose ethers (preferably methylcellulose, ethylcellulose, hydroxyethylcellulose, hydroxypropylcellulose, hydroxypropylmethylcellulose), polyurethanes, polyvinylpyrrolidones, polyamides, polylactides, polyacetals, polylactide/glycolides, polylactones, polyglycolides, polyorthoesters, polyanhydrides, copolymers thereof, block-copolymers thereof, and mixtures of at least two of the stated polymers.

Preferably, the polymer matrix comprises a polymer selected from the group consisting of polyesters (e.g. polylactic acid (PLA) or polyethylene terephthalate (PET)); polyamides; polyurethanes; cellulose ethers (e.g. methylcellulose (MC), ethylcellulose (EC), hydroxypropylcellulose (HPC) and hydroxypropylmethylcellulose (HPMC)); polyacrylates; vinyl polymers (e.g. ethylene vinyl acetate copolymers (EVA), polyvinyl chloride (PVC), polyvinylpyrrolidone (e.g. Kollidon® PF 12) or blends thereof such as polyvinyl acetate/polyvinylpyrrolidone (e.g. Kollidon® SR)); polyether ether ketones; polyalkylene oxides; and mixtures thereof.

In a preferred embodiment, the polymer matrix comprises polyether ether ketone (PEEK).

In a preferred embodiment, the polymer matrix comprises a non-ionic polymer. In another preferred embodiment, the polymer matrix comprises an anionic polymer. In still another preferred embodiment, the polymer matrix comprises a cationic polymer.

Preferably, the polymer is selected from acrylic polymers or polyalkylene oxides.

In a preferred embodiment, the polymer matrix comprises an acrylic polymer which is preferably derived from a monomer mixture comprising a first C₁₋₄-alkyl (meth)acrylate and a second C₁₋₄-alkyl (meth)acrylate differing from said first C₁₋₄-alkyl (meth)acrylate.

Preferred C₁₋₄-alkyl (meth)acrylates include methyl methacrylate, methyl acrylate, ethyl methacrylate, ethyl acrylate, propyl methacrylate, propyl acrylate, butyl methacrylate, and butyl acrylate.

For the purpose of the specification, “(meth)acryl” refers to acryl as well as methacryl.

Preferably, the acrylic polymer has a weight average molecular weight within the range of from 100,000 g/mol to 2,000,000 g/mol. In a preferred embodiment, the acrylic polymer has a weight average molecular weight (M_{W}) or viscosity average molecular weight (M_{η}) of at least 150,000 or at least 200,000 g/mol, preferably at least 250,000 g/mol or at least 300,000 g/mol, more preferably in the range of about 300,000 g/mol to about 2,000,000 g/mol, and most preferably in the range of about 300,000 g/mol to about 1,000,000 g/mol. Suitable methods to determine M_{W} and M_{η} are known to a person skilled in the art. M_{η} is preferably determined by rheological measurements, whereas M_{W} can be determined by gel permeation chromatography (GPC).

The acrylic polymer can be a nonionic acrylic polymer or an ionic acrylic polymer. For the purpose of specification, “nonionic polymer” refers to a polymer not containing more than 1 mole.-% ionic, i.e. anionic or cationic, monomer units, preferably containing no ionic monomer units at all.

In a preferred embodiment, the polymer is a nonionic acrylic polymer.

The nonionic acrylic polymer is preferably derived from a monomer mixture comprising a first C₁₋₄-alkyl (meth)acrylate and a second C₁₋₄-alkyl (meth)acrylate differing from said first C₁₋₄-alkyl (meth)acrylate. Preferably, the first C₁₋₄-alkyl (meth)acrylate is ethyl acrylate and the second C₁₋₄-alkyl (meth)acrylate is methyl methacrylate. Preferably, the relative molar content of the ethyl acrylate within the nonionic acrylic polymer is greater than the relative molar content of the methyl methacrylate within the nonionic acrylic polymer. Preferably, the molar ratio of the first C₁₋₄-alkyl (meth)acrylate, which is preferably ethyl acrylate, to the second C₁₋₄-alkyl (meth)acrylate, which is preferably methyl methacrylate, is within the range of from 5:1 to 1:3, more preferably from 4.5:1 to 1:2.5, still more preferably from 4:1 to 1:2, yet more preferably from 3.5:1 to 1:1.5, even more preferably from 3:1 to 1:1, most preferably from 2.5:1 to 1.5:1, and in particular about 2:1.

The nonionic acrylic polymer may comprise a single nonionic acrylic polymer having a particular average molecular weight, or a mixture (blend) of different nonionic acrylic polymers, such as two, three, four or five nonionic acrylic polymers, e.g., nonionic acrylic polymers of the same chemical nature but different average molecular weight, nonionic acrylic polymers of different chemical nature but same average molecular weight, or nonionic acrylic polymers of different chemical nature as well as different molecular weight.

In a preferred embodiment, the nonionic acrylic polymer is homogeneously distributed in the polymer matrix.

Nonionic acrylic polymers that are suitable for use in the polymer matrix according to the invention are commercially available, e.g. from Evonik. For example, Eudragit® NE30D, Eudragit® NE40D and Eudragit® NM30D, which are provided as aqueous dispersions of poly(ethyl acrylate-co-methyl methacrylate) 2:1, may be used in the polymer matrix according to the invention. For details concerning the properties of these products, it can be referred to e.g. the product specification.

In another preferred embodiment, the polymer is an ionic acrylic polymer.

In a preferred embodiment, the ionic acrylic polymer is homogeneously distributed in the polymer matrix.

Preferred ionic acrylic polymers are anionic acrylic polymers. Preferred anionic acrylic polymers include but are not limited to copolymers of one or two different C₁₋₄-alkyl (meth)acrylate monomers and copolymerizable anionic monomers such as acrylic acid. Preferred representatives are ternary copolymers of methyl acrylate, methyl methacrylate and methacrylic acid, wherein the relative molar content of the monomers is preferably methyl acrylate >methyl methacrylate >methacrylic acid. Preferably, the anionic acrylic polymer has a weight average molecular weight within the range of 280,000±250,000 g/mol, more preferably 280,000±200,000 g/mol, still more preferably 280,000±180,000 g/mol, yet more preferably 280,000±160,000 g/mol, even more preferably 280,000±140,000 g/mol, most preferably 280,000±120,000 g/mol, and in particular 280,000±100,000 g/mol. Poly(methyl acrylate-co-methyl methacrylate-co-methacrylic acid) 7:3:1 having an average molecular weight of about 280,000 g/mol is commercially available as Eudragit® FS.

Other preferred ionic acrylic polymers are cationic acrylic polymers. Preferred cationic acrylic polymers include but are not limited to copolymers of one or two different C₁₋₄-alkyl (meth)acrylate monomers and copolymerizable cationic monomers such as trimethylammonioethyl methacrylate chloride. Preferred representatives are ternary copolymers of ethyl acrylate, methyl methacrylate and a low content of methacrylic acid ester with quaternary ammonium groups, preferably trimethylammonioethyl methacrylate chloride, wherein the relative molar content of the monomers is preferably methyl methacrylate >ethyl acrylate >copolymerizable cationic monomers. Preferably, the cationic acrylic polymer has a weight average molecular weight within the range of 32,000±30,000 g/mol, more preferably 32,000±27,000 g/mol, still more preferably 32,000±23,000 g/mol, yet more preferably 32,000±20,000 g/mol, even more preferably 32,000±17,000 g/mol, most preferably 32,000±13,000 g/mol, and in particular 32,000±10,000 g/mol. Poly(ethyl acrylate-co-methyl methacrylate-co-trimethylammonioethyl methacrylate chloride) 1:2:0.1 and 1:2:0.2, respectively, having an average molecular weight of about 32,000 g/mol is commercially available as Eudragit® RS-PO and Eudragit® RL-PO, respectively. Because of its lower content of trimethylammonioethyl methacrylate chloride, Eudragit® RS-PO is particularly preferred. Another preferred cationic acrylic polymer is Eudragit® RL 100 which is a copolymer of ethyl acrylate, methyl methacrylate and a low content of methacrylic acid ester with quaternary ammonium groups.

In another preferred embodiment, the polymer matrix comprises a polyalkylene oxide, preferably a polyethylene oxide, particularly preferably having an weight average molecular weight of at least 500,000 g/mol.

In a preferred embodiment, the polyalkylene oxide is homogeneously distributed in the polymer matrix.

Preferably, the polyalkylene oxide is selected from polymethylene oxide, polyethylene oxide and polypropylene oxide, or copolymers or mixtures thereof.

Preferably, the polyalkylene oxide has a weight average molecular weight (M_{W}), preferably also a viscosity average molecular weight (M_{η}) of more than 200,000 g/mol or at least 500,000 g/mol, preferably at least 1,000,000 g/mol or at least 2,500,000 g/mol, more preferably in the range of about 1,000,000 g/mol to about 15,000,000 g/mol, and most preferably in the range of about 5,000,000 g/mol to about 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Preferably, the molecular weight dispersity M_{w}/Mₙ of the polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 mPa·s, more preferably 55 to 17,600 mPa·s, still more preferably 600 to 17,600 mPa·s, yet more preferably 4,500 to 17,600 mPa·s, even more preferably 4,500 to 12,000 mPa·s, most preferably 5,000 to 10,500 mPa·s and in particular 5,500 to 7,500 mPa·s or 7,500 to 10,000 mPa·s, measured in a 1 wt.-% aqueous solution.

The polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of specification, a polyalkylene glycol has a molecular weight of up to 20,000 g/mol whereas a polyalkylene oxide has a molecular weight of more than 20,000 g/mol. The weight average over all molecular weights of all polyalkylene oxides that are contained in the pharmaceutical dosage form is more than 200,000 g/mol. Thus, polyalkylene glycols, if any, are preferably not taken into consideration when determining the weight average molecular weight of polyalkylene oxide.

In a particularly preferred embodiment, the polymer is a polyalkylene oxide the content of which is at least 30 wt.-% relative to the total weight of the polymer matrix.

Preferably, the polyalkylene oxide is combined with another polymer, preferably a cellulose ether, particularly preferably a cellulose ether selected from the group consisting of methylcellulose, ethylcellulose, hydroxyethylcellulose, hydroxypropylcellulose, and hydroxypropylmethylcellulose. Hydroxypropylmethylcellulose is particularly preferred.

Preferably, the relative weight ratio of the polyalkylene oxide and the cellulose ether is within the range of from 14:1 to 1:2, more preferably 13:1 to 1:1, still more preferably 12:1 to 2:1, yet more preferably 11:1 to 3:1, even more preferably 10:1 to 4:1, most preferably 9:1 to 5:1, and in particular 8:1 to 6:1.

Preferably, the weight content of the polymer matrix is within the range of from 5.0 to 95 wt.-%, more preferably 10 to 90 wt.-%, still more preferably 25 to 85 wt.-%, relative to the total weight of the pharmaceutical dosage form.

In preferred embodiments, the weight content of the polymer matrix is within the range of from 10±5 wt.-%, 20±15 wt.-%, or 20±10 wt.-%, or 20±5 wt.-%, or 30±25 wt.-%, or 30±20 wt.-%, or 30±15 wt.-%, or 30±10 wt.-%, or 30±5 wt.-%, or 40±35 wt.-%, or 40±30 wt.-%, or 40±25 wt.-%, or 40±20 wt.-%, or 40±15 wt.-%, or 40±10 wt.-%, or 40±5 wt.-%, or 50±45 wt.-%, or 50±40 wt.-%, or 50±35 wt.-%, or 50±30 wt.-%, or 50±25 wt.-%, or 50±20 wt.-%, or 50±15 wt.-%, or 50±10 wt.-%, or 50±5 wt.-%, or 60±35 wt.-%, or 60±30 wt.-%, or 60±25 wt.-%, or 60±20 wt.-%, or 60±15 wt.-%, or 60±10 wt.-%, or 60±5 wt.-%, or 70±25 wt.-%, or 70±20 wt.-%, or 70±15 wt.-%, or 70±10 wt.-%, or 70±5 wt.-%, or 80±15 wt.-%, or 80±10 wt.-%, or 80±5 wt.-%, or 90±5 wt.-%, relative to the total weight of the pharmaceutical dosage form.

Preferably, the polymer matrix is manufactured by three-dimensional printing technology. More preferably, the three-dimensional printing technology is fused deposition modeling.

The fibers of the pharmaceutical dosage form according to the invention are not particularly limited. Principally, every fibers can be used that are conventionally used for the manufacture of reinforced materials and that are not harmful to the organism.

In a preferred embodiment, the pharmaceutical dosage form according to the invention comprises a reinforced polymer matrix comprising or essentially consisting of one or more non-erodible polymers. Under these circumstances, the polymer matrix comprising the fibers is excreted as such, i.e. in a non-eroded state, and thus the fibers are not released from the polymer matrix after ingestion. Thus, no harm may be caused by the fibers and the scope of fibers that are suitable according to the invention is therefore very broad.

In another preferred embodiment, the pharmaceutical dosage form according to the invention comprises fibers that in turn comprise or essentially consist of one or more non-erodible polymers.

Preferably, the fibers are selected from the group consisting of glass fibers, carbon fibers, mineral fibers, polymer fibers, and mixtures thereof.

In a preferred embodiment, the fibers are made of a material, e.g. of a polymer or polymer blend, that has elevated hardness at room temperature. Preferably, the fibers are made of a material that at room temperature has a shore hardness D (in accordance with DIN ISO 7619-1) of at least 40, or at least 42.5, or at least 45, or at least 47.5; more preferably at least 50, or at least 52.5, or at least 55, or at least 57.5; still more preferably at least 60, or at least 62.5, or at least 65, or at least 67.5; yet more preferably at least 70, or at least 72.5, or at least 75, or at least 77.5; even more preferably at least 80, or at least 81, or at least 82, or at least 83, or at least 84; most preferably at least 85, or at least 86, or at least 87, or at least 88, or at least 89; and in particular at least 90, or at least 91, or at least 92, or at least 93, or at least 94, or at least 95.

In a preferred embodiment of pharmaceutical dosage form according to the invention the fibers are polymer fibers comprising a polymer selected from the group consisting of polyesters (e.g. polylactic acid (PLA) or polyethylene terephthalate (PET)); polyamides; polyurethanes; cellulose ethers (e.g. methylcellulose (MC), ethylcellulose (EC), hydroxypropylcellulose (HPC) and hydroxypropylmethylcellulose (HPMC)); polyacrylates; vinyl polymers (e.g. ethylene vinyl acetate copolymers (EVA), polyvinyl chloride (PVC), polyvinylpyrrolidone (e.g. Kollidon® PF 12) or blends thereof such as polyvinyl acetate/polyvinylpyrrolidone (e.g. Kollidon® SR)); polyether ether ketones; polyalkylene oxides; and mixtures thereof.

In a particularly preferred embodiment, the fibers comprise or essentially consist of polyamide, more preferably of aramid (e.g. Kevlar®, Nomex® and Technora®).

The weight content of the fibers in the pharmaceutical dosage form according to the invention is not particularly limited.

In a preferred embodiment, particularly when the pharmaceutical dosage form comprises a polymer matrix comprising the fibers, the weight content of the fibers is within the range of from 0.1 to 50 wt.-%, more preferably 0.1 to 20 wt.-%, relative to the total weight of the pharmaceutical dosage form.

In another preferred embodiment, particularly when the fibers as such comprise or essentially consist of one or more polymers and are preferably of macroscopic size, the weight content of the fibers is within the range of from 5.0 to 80 wt.-%, more preferably 10 to 60 wt.-%, relative to the total weight of the pharmaceutical dosage form.

In preferred embodiments, the weight content of the fibers is within the range of from 10±5 wt.-%, 20±15 wt.-%, or 20±10 wt.-%, or 20±5 wt.-%, or 30±25 wt.-%, or 30±20 wt.-%, or 30±15 wt.-%, or 30±10 wt.-%, or 30±5 wt.-%, or 40±35 wt.-%, or 40±30 wt.-%, or 40±25 wt.-%, or 40±20 wt.-%, or 40±15 wt.-%, or 40±10 wt.-%, or 40±5 wt.-%, or 50±45 wt.-%, or 50±40 wt.-%, or 50±35 wt.-%, or 50±30 wt.-%, or 50±25 wt.-%, or 50±20 wt.-%, or 50±15 wt.-%, or 50±10 wt.-%, or 50±5 wt.-%, or 60±35 wt.-%, or 60±30 wt.-%, or 60±25 wt.-%, or 60±20 wt.-%, or 60±15 wt.-%, or 60±10 wt.-%, or 60±5 wt.-%, or 70±25 wt.-%, or 70±20 wt.-%, or 70±15 wt.-%, or 70±10 wt.-%, or 70±5 wt.-%, or 80±15 wt.-%, or 80±10 wt.-%, or 80±5 wt.-%, or 90±5 wt.-%, relative to the total weight of the pharmaceutical dosage form.

The fibers according to the invention are preferably of macroscopic size. A fiber according to the invention is not to be interpreted on a molecular level, i.e. natural or synthetic polymeric (macro)molecules as such, like e.g. cellulose molecules, polyalkylene oxides molecules, and the like are not to be regarded as fibers according to the invention. Further, strands, helices, fibrils or microfibrils which are formed of a plurality of such natural polymeric (macro)molecules, e.g. protein based structures like collagen or cellulosic structures such as microcrystalline cellulose or xanthan gum, are also not to be regarded as fibers according to the invention. Typically, the average diameter of such strands, helices, fibrils or microfibrils is in the range of several nm only.

The cellulosic components of a wood fiber wall structure are the cellulose molecule, the elementary fibril, the microfibril, the macrofibril and the lamellar membrane. The term “elementary fibril” was reported to have a diameter of 3.5 nm. Elementary fibrils with diameters of approximately 3.5 nm also occur in cotton and bacterial cellulose. Thus, due to their small size, such cellulosic components are not to be regarded as fibers according to the invention.

Carboxymethylcellulose forms rather flexible structures with alternating thin and thick segments within the nanofibers with diameters ranging from 10 to 16 nm and a length of up to 1 μm. Hyaluronate, a high-molecular-mass molecule, forms extra-long aggregates of more than 5 μm. Individual fibers with a diameter of 8 nm aggregated to bigger strands. The nonlinear polysaccharide xanthan gum leads to highly coiled structures. The diameter of the respective nanofibers varies between 15 and 25 nm. Thus, due to their small size, such structures of carboxymethylcellulose, hyaluronate, xanthan gum are not to be regarded as fibers according to the invention.

The fundamental structural unit of fibrous type I collagen is a long (300-nm), thin (1.5-nm-diameter) protein that consists of three coiled subunits: two α1(I) chains and one α2(I). Each chain contains precisely 1050 amino acids wound around one another in a characteristic right-handed triple helix. All collagens were eventually shown to contain three-stranded helical segments of similar structure; the unique properties of each type of collagen are due mainly to segments that interrupt the triple helix and that fold into other kinds of three-dimensional structures. Thus, due to their small size, such structures of collagen are not to be regarded as fibers according to the invention.

The dimensions of the fibers are not particularly limited. The pharmaceutical dosage form may comprise fibers of substantially different dimensions. Preferably, however, the pharmaceutical dosage form comprises fibers of essentially identical dimensions within the limits of size distribution that may be caused by the various processes for the preparation of fibers.

In a preferred embodiment, particularly when the pharmaceutical dosage form comprises a polymer matrix comprising the fibers, the fibers have an average diameter of
at least 0.1 μm, more preferably at least 0.5 μm, still more preferably at least 1.0 m; and/or
at most 250 μm, more preferably at most 200 μm, still more preferably at most 150 μm.

In another preferred embodiment, particularly when the fibers as such comprise or essentially consist of one or more polymers and are preferably of macroscopic size, the fibers have an average diameter of
at least 2.0 μm, more preferably at least 5.0 μm, still more preferably at least 10 μm; and/or
at most 2.5 mm, more preferably at most 2.0 mm, still more preferably at most 1.5 mm.

In preferred embodiments, the fibers have an average diameter within the range of 2.5±2.0 μm, or 2.5±1.5 μm, or 2.5±1.0 μm, or 2.5±0.5 μm, or 5.0±4.5 μm, or 5.0±4.0 μm, or 5.0±3.5 μm, or 5.0±3.0 μm, or 10±9 μm, or 10±8 μm, or 10±7 μm, or 10±6 μm, or 10±5 μm, or 25±20 μm, or 25±15 μm, or 25±10 μm, or 25±5 μm, or 50±45 μm, or 50±40 μm, or 50±35 μm, or 50±30 μm, or 100±90 μm, or 100±80 μm, or 100±70 μm, or 100±60 μm, or 100±50 μm, or 250±200 μm, or 250±150 μm, or 250±100 μm, or 250±50 μm, or 500±450 μm, or 500±400 μm, or 500±350 μm, or 500±300 μm, or 1000±900 μm, or 1000±800 μm, or 1000±700 μm, or 1000±600 μm, or 1000±500 μm.

In a preferred embodiment, particularly when the pharmaceutical dosage form comprises a polymer matrix comprising the fibers, the fibers have an average length of
at least 1.0 μm, more preferably at least 5.0 μm, still more preferably at least 10 μm; and/or
at most 2500 μm, more preferably at most 2000 μm, still more preferably at most 1500 μm.

In another preferred embodiment, particularly when the fibers as such comprise or essentially consist of one or more polymers and are preferably of macroscopic size, the fibers have an average length of
at least 20 μm, more preferably at least 50 μm, still more preferably at least 100 μm; and/or
at most 25 mm, more preferably at most 20 mm, still more preferably at most 15 mm.

Preferably, the fibers have an average length of at least 1100 μm, or of at least 1200 μm, or of at least 1300 μm, or of at least 1400 μm, or of at least 1500 μm, or of at least 1600 μm, or of at least 1700 μm, or of at least 1800 μm, or of at least 1900 μm, or of at least 2000 μm, or of at least 2100 μm, or of at least 2200 μm, or of at least 2300 μm, or of at least 2400 μm, or of at least 2500 μm, or of at least 2600 μm, or of at least 2700 μm, or of at least 2800 μm, or of at least 2900 μm, or of at least 3000 μm.

In preferred embodiments, the fibers have an average length within the range of 2.5±2.0 μm, or 2.5±1.5 μm, or 2.5±1.0 μm, or 2.5±0.5 μm, or 5.0±4.5 μm, or 5.0±4.0 μm, or 5.0±3.5 μm, or 5.0±3.0 μm, or 10±9 μm, or 10±8 μm, or 10±7 μm, or 10±6 μm, or 10±5 μm, or 25±20 μm, or 25±15 μm, or 25±10 μm, or 25±5 μm, or 50±45 μm, or 50±40 μm, or 50±35 μm, or 50±30 μm, or 100±90 μm, or 100±80 μm, or 100±70 μm, or 100±60 μm, or 100±50 μm, or 250±200 μm, or 250±150 μm, or 250±100 μm, or 250±50 μm, or 500±450 μm, or 500±400 μm, or 500±350 μm, or 500±300 μm, or 1000±900 μm, or 1000±800 μm, or 1000±700 μm, or 1000±600 μm, or 10000±5000 μm, or 2500±2000 μm, or 2500±1500 μm, or 2500±1000 μm, or 2500±500 μm, or 5000±4500 μm, or 5000±4000 μm, or 5000±3500 μm, or 5000±3000 μm, or 10000±9000 μm, or 10000±8000 μm, or 10000±7000 μm, or 10000±6000 μm, or 10000±5000 μm.

Preferably, the fibers have an average aspect ratio
of at least 2.5, or of at least 3.0, or of at least 3.5, or of at least 4.0, or of at least 4.5, more preferably of at least 5.0, or at least 5.5, or of at least 6.0, or of at least 6.5, or of at least 7.0, or of at least 7.5, or of at least 8.0, or of at least 9.0, or of at least 9.5, even more preferably of at least 10.0, or of at least 11.0, or of at least 12.0, or of at least 13.0, or of at least 14.0, or of at least 15.0, or of at least 16.0, or of at least 17.0, or of at least 18.0, or of at least 19.0, or of at least 10.0, or of at least 20.0, or of at least 30.0, or of at least 40.0, or of at least 50.0, or of at least 60.0, or of at least 70.0, or of at least 80.0, or of at least 90.0, or of at least 100.0; and/or
of at most 250, or of at most 245, or of at most 240, or of at most 235, or of at most 230, more preferably of at most 225, or of at most 220, or of at most 215, or of at most 210, or of at most 205, or of at most 200, or of at most 190, or of at most 180, or of at most 170, or of at most 160, or of at most 150, or of at most 140, or of at most 130, or of at most 120, or of at most 110.

In preferred embodiments, the fibers have an average aspect ratio within the range of 5.0±4.5, or 5.0±4.0, or 5.0±3.5, or 5.0±3.0, or 10±9, or 10±8, or 10±7, or 10±6, or 10±5, or 25±20, or 25±15, or 25±10, or 25±5, or 50±45, or 50±40, or 50±35, or 50±30, or 75±70, or 75±65, or 75±60, or 75±55, or 100±90, or 100±80, or 100±70, or 100±60, or 100±50.

Preferably, at least 10 wt.-% of the fibers comprised in the dosage form, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-% of the fibers comprised in the dosage form have essentially identical dimensions within the limits of size distribution that may be caused by the preparation process of the fibers.

In a preferred embodiment, the fibers do not comprise the pharmacologically active ingredient.

In another preferred embodiment, the fibers comprise at least 10 wt.-%, or at least 20 wt.-%, or at least 30 wt.-%, more preferably at least 40 wt.-%, or at least 50 wt.-%, or at least 60 wt.-%, even more preferably at least 70 wt.-%, or at least 80 wt.-%, or at least 90 wt.-% of the pharmacologically active ingredient comprised in the dosage form.

In a preferred embodiment, the fibers are manufactured by three-dimensional printing technology, preferably the three-dimensional printing technology is fused deposition modeling.

The nature of the pharmacologically active ingredient that is contained in the pharmaceutical dosage form is not particularly limited. The pharmaceutical dosage form may comprise a single pharmacologically active ingredient or a combination of two or more pharmacologically active ingredients.

Preferably, the pharmacologically active ingredient has psychotropic action. Preferably, the pharmacologically active ingredient is selected from opioids and stimulants.

Preferably, the pharmacologically active ingredient is selected from ATC class [N], more preferably [N02] according to the WHO.

Particularly preferably, the pharmacologically active ingredient is an opioid. For the purpose of specification, the term “opioid” shall refer to any opioid as well as any physiologically acceptable salt thereof. Thus, preferably, the dosage form comprises an opioid or a physiologically acceptable salt thereof.

According to the ATC index, opioids are divided into natural opium alkaloids, phenylpiperidine derivatives, diphenylpropylamine derivatives, benzomorphan derivatives, oripavine derivatives, morphinan derivatives and others. In a preferred embodiment, the pharmacologically active ingredient is selected from the group consisting of morphine, hydromorphone, nicomorphine, oxycodone, oxymorphone, dihydrocodeine, ketobemidone, pethidine, fenantyl, dextromoramide, piritramide, dextropropoxyphene, bezitramide, pentazocine, phenazocine, buprenorphine, butorphanol, nalbuphine, tilidine, tramadol, dezocine, meptazinol, tapentadol, and the physiologically acceptable salts thereof.

In a particularly preferred embodiment, the pharmacologically active ingredient is selected from the group consisting of oxycodone, oxymorphone, hydrocodone, hydromorphone, tramadol, tapentadol, morphine, buprenorphine and the physiologically acceptable salts thereof.

In yet another preferred embodiment, the pharmacologically active ingredient is selected from the group consisting of 1,1-(3-dimethylamino-3-phenylpentamethylene)-6-fluoro-1,3,4,9-tetrahydropyrano[3,4-b]indole; 1,1-[3-dimethylamino-3-(2-thienyl)pentamethylene]-1,3,4,9-tetrahydropyrano[3,4-b]indole; and 1,1-[3-dimethylamino-3-(2-thienyl)pentamethylene]-1,3,4,9-tetrahydropyrano[3,4-b]-6-fluoroindole. These compounds are known from, e.g., WO 2004/043967, WO 2005/066183.

Preferably, the pharmacologically active ingredient is selected from the following compounds: alfentanil, allylprodine, alphaprodine, apocodeine, axomadol, bemidone, benzylmorphine, bezitramide, buprenorphine, butorphanol, carfentanil, clonitazene, cocaine, codeine, cyclorphan, cyprenorphine, desomorphine, dextromoramide, dextropropoxyphene, dezocine, diampromide, diamorphone, dihydrocodeine, dihydromorphine, dihydromorphone, dimenoxadol, dimephetamol, dimethylthiambutene, dioxaphetylbutyrate, dipipanone, eptazocine, ethoheptazine, ethylmethylthiambutene, ethylmorphine, etonitazene, etorphine, faxeladol, fentanyl, heroin, hydrocodone, hydromorphone, hydroxypethidine, isomethadone, hydroxymethylmorphinan, ketobemidone, levacetylmethadol (LAAM), levomethadone, levorphanol, levophenacylmorphane, lofentanil, meperidine, metapon, meptazinol, metazocine, methylmorphine, methadone, 3-methylfentanyl, 4-methylfentanyl, metopon, morphine, myrophine, nalbuphine, nalorphine, narceine, nicomorphine, norlevorphanol, normethadone, normorphine, norpipanone, opium, oxycodone, oxymorphone, Papaver somniferum, papaveretum, pentazocine, pethidine, phenadoxone, phenomorphane, phenazocine, phenoperidine, piminodine, pholcodeine, piritramide, profadol, proheptazine, promedol, properidine, propoxyphene, remifentanil, sufentanil, tapentadol, tilidine (cis and trans), tramadol, N-(1-methyl-2-piperidinoethyl)-N-(2-pyridyl)propionamide, (1R,2R)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutyl-phenyl)propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl)propionate, (RR-SS)-2-acetoxy-4-trifluoromethyl-benzoic acid 3-(2-dimethylaminomethyl-1-hydroxy-cyclohexyl)-phenyl ester, (RR-SS)-2-hydroxy-4-trifluoro-methyl-benzoic acid 3-(2-dimethylaminomethyl-1-hydroxy-cyclohexyl)-phenyl ester, (RR-SS)-4-chloro-2-hydroxy-benzoic acid 3-(2-dimethylaminomethyl-1-hydroxy-cyclohexyl)-phenyl ester, (RR-SS)-2-hydroxy-4-methyl-benzoic acid 3-(2-dimethylaminomethyl-1-hydroxy-cyclohexyl)-phenyl ester, (RR-SS)-2-hydroxy-4-methoxy-benzoic acid 3-(2-dimethylaminomethyl-1-hydroxy-cyclohexyl)-phenyl ester, (RR-SS)-2-hydroxy-5-nitro-benzoic acid 3-(2-dimethylaminomethyl-1-hydroxy-cyclohexyl)-phenyl ester, (RR-SS)-2′,4′-difluoro-3-hydroxy-biphenyl-4-carboxylic acid 3-(2-dimethylaminomethyl-1-hydroxy-cyclohexyl)-phenyl ester, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

In another preferred embodiment, the pharmacologically active ingredient is selected from the group consisting of DPI-125, M6G (CE-04-410), ADL-5859, CR-665, NRP290 and sebacoyl dinalbuphine ester.

In another preferred embodiment, the pharmacologically active ingredient is selected from the group consisting of rabeprazole, fentanyl, risedronate, nifedipine, amphetamine salts, everolimus, alprazolam, lovastatin, zolpidem, dalfampridine, cyclobenzaprine, bupropion, mesalamine, tipranavir, donepezil, diclofenac, aspirin, sulfasalazine, morphine, dutasteride, clarithromycin, praziquantel, bisacodyl, ibandronate, verapamil, nicardipine, diltiazem, doxazosin, cefuroxime, mycophenolate, activated charcoal, ciprofloxacin, docusate, colestipol, methylphenidate, nicotine, carvedilol, pancrelipase, indinavir, duloxetine, cyclophosphamide, ganciclovir, divalproex, tolterodine, dexlansoprazole, doxylamine, pyridoxine, diltiazem, isosorbide, oxybutynin, ergocalciferol, hydroxyurea, isradipine, erythromycin, potassium bicarbonate, venlafaxine, morphine sulfate, darifenacin, budesonide, ergotamine, vismodegib, raloxifene, hydromorphone, deferasirox, piroxicam, fentanyl, ferrous sulfate, ferrous gluconate, metronidazole, tamsulosin, dexmethylphenidate, metformin, alendronate, imatinib, glipizide, gabapentin, propranolol, indomethacin, etravirine, zolpidem, guanfacine, paliperidone, isotretinoin, ruxolitinib, dutasteride, tamsulosin, sitagliptin, lopinavir, ritoavir, dexlansoprazole, clonidine, alogliptin, levetiracetam, telithromycin, desvenlafaxine, potassium salt, lamotrigine, fluvastatin, ambrisentan, hyoscyamine, lithium salt, brompheniramine, fluvoxamine, pyridostigmine, potassium chloride, pramipexole, amoxicillin, ibuprofen, guiafenesin, mycophenolate, mirabegron, memantine, naproxen, esomeprazole, nicotinic acid, nifedipine, nitroglycerin, orphenadrine, disopyramide, ritonavir, posaconazole, tapentadole, trazodone, doxycycline, oxycodone, pancrealipase, paroxetine, dabigatran, felodipide, lansoprazole, omeprazole, finasteride, ciprofloxicin, pantoprazole, fluoxetine, renolazine, sirolimus, prednisone, galantamine, sevelamer, sevelamer carbonate, ropinirole, lenalidomide, propafenone, tramadol, cinacalcet, quetiapine, levodopa, carbidopa, minocycline, chloral hydrate, dasatinib, atomoxetine, nisoldipine, hyoscyamine, nilotinib, diltiazem, dimethyl fumarate, carbamazepine, temozolomide, benzonatate, theophylline, topiramate, metoprolol, fesoterodine, bosentan, pentoxifylline, fenofibric, acetaminophen, budesonide, potassium citrate, alfuzosin, valganciclovir, didanosine, naproxen, esomeprazole, nevirapine, albuterol, pazopanib, rivaroxaban, omeprazole/NaHCO₃, hydrocodone, vorinostat, everolimus, zileuton, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

The pharmacologically active ingredient may be present in form of a physiologically acceptable salt, e.g. physiologically acceptable acid addition salt.

Physiologically acceptable acid addition salts comprise the acid addition salt forms which can conveniently be obtained by treating the base form of the pharmacologically active ingredient with appropriate organic and inorganic acids. Pharmacologically active ingredients containing an acidic proton may be converted into their non-toxic metal or amine addition salt forms by treatment with appropriate organic and inorganic bases. The term addition salt also comprises the hydrates and solvent addition forms which the active ingredients are able to form. Examples of such forms are e.g. hydrates, alcoholates and the like.

The pharmacologically active ingredient is present in the dosage form in a therapeutically effective amount. The amount that constitutes a therapeutically effective amount varies according to the pharmacologically active ingredients being used, the condition being treated, the severity of said condition, the patient being treated, and the frequency of administration.

The absolute content of the pharmacologically active ingredient in the dosage form is not limited. The dose of the pharmacologically active ingredient preferably is in the range of 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the pharmacologically active ingredient, preferably the opioid that is contained in the dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

Preferably, the weight content of the pharmacologically active ingredient is within the range of from 0.01 to 80 wt.-%, more preferably 0.1 to 50 wt.-%, still more preferably 5.0 to 50 wt.-%, yet more preferably 1 to 35 wt.-%, based on the total weight of the dosage form.

In preferred embodiments, the weight content of the pharmacologically active ingredient, preferably the opioid is within the range of from 5.0±4.5 wt.-%, or 10±9.0 wt.-%, or 15±14 wt.-%, or 20±19 wt.-%, or 25±24 wt.-%; more preferably 5.0±4.0 wt.-%, or 10±8.0 wt.-%, or 15±12 wt.-%, or 20±19 wt.-%, or 25±24 wt.-%; still more preferably 5.0±3.5 wt.-%, or 10±7.0 wt.-%, or 15±10 wt.-%, or 20±17 wt.-%, or 25±21 wt.-%; yet more preferably 5.0±3.0 wt.-%, or 10±6.0 wt.-%, or 15±8.0 wt.-%, or 20±15 wt.-%, or 25±18 wt.-%; even more preferably 5.0±2.5 wt.-%, or 10±5.0 wt.-%, or 15±6.0 wt.-%, or 20±13 wt.-%, or 25±15 wt.-%; most preferably 5.0±2.0 wt.-%, or 10±4.0 wt.-%, or 15±4.0 wt.-%, or 20±11 wt.-%, or 25±12 wt.-%; and in particular 5.0±1.5 wt.-%, or 10±3.0 wt.-%, or 15±2.0 wt.-%, or 20±9 wt.-%, or 25±9 wt.-%; in each case either based on the total weight of the dosage form.

The skilled person may readily determine an appropriate amount of pharmacologically active ingredient, preferably opioid to include in a dosage form. For instance, in the case of analgesics, the total amount of pharmacologically active ingredient, preferably opioid present in the dosage form is that sufficient to provide analgesia. The total amount of pharmacologically active ingredient, preferably opioid administered to a patient in a dose will vary depending on numerous factors including the nature of the pharmacologically active ingredient, the weight of the patient, the severity of the pain, the nature of other therapeutic agents being administered etc.

In a preferred embodiment, the pharmaceutical dosage form according to the invention under in vitro conditions provides rapid release of the pharmacologically active ingredient such that after 30 minutes in artificial gastric juice it has released at least 50 wt.-%, more preferably at least 80 wt.-% of the pharmacologically active ingredient that was original contained in the pharmaceutical dosage form.

In another preferred embodiment, the pharmaceutical dosage form according to the invention under in vitro conditions provides prolonged release of the pharmacologically active ingredient such that after 30 minutes in artificial gastric juice it has released less than 50 wt.-%, more preferably less than 30 wt.-% of the pharmacologically active ingredient that was original contained in the pharmaceutical dosage form.

Preferably, under in vitro conditions in 900 mL artificial (simulated) gastric fluid (pH 1.2 HCl) in accordance with Ph. Eur. paddle method, at 50 rpm and 37° C., the pharmaceutical dosage form according to the invention exhibits a release profile according to any of embodiments A¹ to A⁸ as compiled in the table here below:

| | | A¹ | A² | A³ | A⁴ | A⁵ | A⁶ | A⁷ | A⁸ |
|---|---|---|---|---|---|---|---|---|---|
| 30 | min | ≥5% | ≥5% | ≥5% | ≥5% | ≥5% | ≥5% | ≥5% | ≥5% |
| 60 | min | ≥10% | ≥10% | ≥10% | ≥10% | ≥10% | ≥10% | ≥10% | ≥10% |
| 2 | h | 15-70% | 20-65% | 25-60% | 30-55% | 15-60% | 20-55% | 25-50% | 30-45% |
| 4 | h | ≤75 | ≤70 | ≤65 | ≤60 | 20-65% | 25-50% | 30-45% | 35-40% |
| 6 | h | ≤80% | ≤80% | ≤80% | ≤80% | 25-70% | 30-65% | 35-60% | 40-55% |
| 9 | h | ≥80% | ≥80% | ≥80% | ≥80% | ≤75 | ≤70 | ≤65 | ≤60 |
| 12 | h | ≥95% | ≥95% | ≥95% | ≥95% | ≤80% | ≤80% | ≤80% | ≤80% |
| 18 | h | ≥95% | ≥95% | ≥95% | ≥95% | ≥80% | ≥80% | ≥80% | ≥80% |
| 24 | h | ≥95% | ≥95% | ≥95% | ≥95% | ≥95% | ≥95% | ≥95% | ≥95% |

Preferably, the pharmaceutical dosage form according to the invention is tamper resistant.

As used herein, the term “tamper-resistant” refers to dosage forms or segments that are resistant to conversion into a form suitable for misuse or abuse, particular for nasal and/or intravenous administration, by conventional means.

Preferably, it provides resistance against mechanical disruption, especially against breaking and/or against cutting, and/or solvent extraction. In a preferred embodiment, the dosage form further provides resistance against solvent extraction and/or resistance against grinding.

Preferably, tamper resistance means that the dosage form
(i) provides resistance against dose-dumping in aqueous ethanol; and/or
(ii) preferably provides resistance against solvent extraction; and/or
(iii) preferably provides resistance against grinding.

Thus, the dosage form does not necessarily need to exhibit resistances (i), (ii) and (iii) simultaneously; but may preferably exhibit only (i), or only (ii), or only (iii), or a combination thereof; namely a combination of only (i) and (ii); a combination of only (i) and (iii); a combination of (ii) and (iii); or a combination of (i) and (ii) and (iii).

In a preferred embodiment, the dosage form according to the invention has a breaking strength of at least 200 N, more preferably at least 300 N. According to this embodiment, the dosage form preferably has a breaking strength of at least 300 N, at least 400 N, or at least 500 N, preferably at least 600 N, more preferably at least 700 N, still more preferably at least 800 N, yet more preferably at least 1000 N, most preferably at least 1250 N and in particular at least 1500 N. Further according to this embodiment, preferably, the dosage form cannot be pulverized by the application of force with conventional means, such as for example a pestle and mortar, a hammer, a mallet or other usual means for pulverization, in particular devices developed for this purpose (dosage form crushers). In this regard “pulverization” means crumbling into small particles. Avoidance of pulverization virtually rules out oral or parenteral, in particular intravenous or nasal abuse.

The “breaking strength” (resistance to crushing) of a dosage form is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Pharmaceutical dosage forms, Vol. 2, Informa Healthcare; 2 edition, 1990; and Encyclopedia of Pharmaceutical Technology, Informa Healthcare; 1 edition.

For the purpose of specification, the breaking strength is preferably defined as the amount of force that is necessary in order to fracture a dosage form (=breaking force). Therefore, for the purpose of specification, a dosage form does preferably not exhibit the desired breaking strength when it breaks, i.e., is fractured into at least two independent parts that are separated from one another. In another preferred embodiment, however, the dosage form is regarded as being broken if the force decreases by 25% (threshold value) of the highest force measured during the measurement (see below).

Methods for measuring the breaking strength are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Pharmaceutical dosage forms”. The test is intended to determine, under defined conditions, the resistance to crushing of dosage forms measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the dosage form. The apparatus is calibrated using a system with a precision of 1 Newton. The dosage form is placed between the jaws, taking into account, where applicable, the shape, the break-mark and the inscription; for each measurement the dosage form is oriented in the same way with respect to the direction of application of the force (and the direction of extension in which the breaking strength is to be measured). The measurement is carried out on 10 dosage forms taking care that all fragments have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a dosage form to fail (i.e., break) in a specific plane. The dosage forms are generally placed between two platens, one of which moves to apply sufficient force to the dosage form to cause fracture. For conventional, round (circular cross-section) dosage forms loading occurs across their diameter (sometimes referred to as diametral loading), and fracture occurs in the plane. The breaking force of dosage forms is commonly called hardness in the pharmaceutical literature; however, the use of this term is misleading. In material science, the term hardness refers to the resistance of a surface to penetration or indentation by a small probe. The term crushing strength is also frequently used to describe the resistance of dosage forms, to the application of a compressive load. Although this term describes the true nature of the test more accurately than does the term hardness, it implies that dosage forms are actually crushed during the test, which is often not the case.

Alternatively, the breaking strength (resistance to crushing) can be measured in accordance with WO 2008/107149, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

In a preferred embodiment, the dosage form is regarded as being broken if it is fractured into at least two separate pieces.

In a preferred embodiment, the dosage form according to the invention provides improved cut resistance. The cut resistance is preferably evaluated in accordance with the test conditions of EN ISO 13997 or ASTM F1790. The EN ISO 13997 test uses the principle of a straight blade drawn across the sample material at a constant speed and weight. The distance travelled to cause cut through is then recorded and the results are calculated to give the force required to cut through at 20 mm of blade travel. For smaller pharmaceutical dosage forms, the blade travel is preferably reduced to 10 mm or 5.0 mm, respectively. Preferably, the thus determined cut resistance of the pharmaceutical dosage form according to the invention is at least 20 N, or at least 25 N, or at least 30 N, or at least 35 N, or at least 40 N, or at least 45 N, or at least 50 N, or at least 75 N, or at least 100 N, or at least 150 N, or at least 200 N, or at least 250 N.

In a preferred embodiment, the dosage form according to the invention provides tamper resistance in terms of resistance against extraction of the pharmacologically active ingredient from the pharmaceutical dosage form in organic solvents. Preferred organic solvents include but are not limited to ethanol, grain alcohol, gasoline, light gas, and the like.

In a preferred embodiment, the dosage form according to the invention provides tamper resistance in terms of resistance against dose-dumping in aqueous ethanol.

The dosage form can be tested in vitro using 0.1 N HCl with 40 vol.-% ethanol to evaluate alcohol extractability. Testing is preferably performed using standard procedures, e.g. USP Apparatus 1 (basket) or USP Apparatus 2 (paddle) at e.g. 50 rpm in e.g. 900 mL of media at 37° C., using a Perkin Elmer UV/VIS Spectrometer Lambda 20, UV at an appropriate wavelength for detection of the pharmacologically active ingredient present therein. Sample time points preferably include 0.5 and 1 hour.

Preferably, when comparing the in vitro release profile at 37° C. in 0.1 N HCl with the in vitro release profile in 0.1 N HCl/ethanol (40 vol.-%) at 37° C., the in vitro release 0.1 N HCl/ethanol (40 vol.-%) is preferably not substantially accelerated compared to the in vitro release in 0.1 N HCl. Preferably, in this regard “substantially” means that at any given time point the in vitro release in 0.1 N HCl/ethanol (40 vol.-%) relatively deviates from the in vitro release in 0.1 N HCl by not more than +15%, more preferably not more than +10%, still more preferably not more than +8%, yet more preferably not more than +6%, even more preferably not more than +4%, most preferably not more than +2% and in particular not more than +1% or not more than +0.5% or not more than +0.1%.

Preferably, with the dosage forms according to the invention, a substantial relative deceleration of the in vitro release in 0.1 N HCl/ethanol (40 vol.-%) compared to the in vitro release in 0.1 N HCl is observed. In a particularly preferred embodiment, at any given time point the in vitro release in 0.1 N HCl/ethanol (40 vol.-%) relatively deviates from the in vitro release in 0.1 N HCl by at least −0.01%, more preferably at least −0.05%, still more preferably at least −0.1%, most preferably at least −0.5% and in particular at least −1%.

Further, the dosage form can be tested in vitro using ethanol/simulated gastric fluid of 0%, 20% and 40% to evaluate alcohol extractability. Testing is preferably performed using standard procedures, e.g. USP Apparatus 1 (basket) or USP Apparatus 2 (paddle) at e.g. 50 rpm in e.g. 900 mL of media at 37° C., using a Perkin Elmer UV/VIS Spectrometer Lambda 20, UV at an appropriate wavelength for detection of the pharmacologically active ingredient present therein. Sample time points preferably include 0.5 and 1 hour.

Preferably, when comparing the in vitro release profile at 37° C. in simulated gastric fluid with the in vitro release profile in ethanol/simulated gastric fluid (40 vol.-%) at 37° C., the in vitro release in ethanol/simulated gastric fluid (40 vol.-%) is preferably not substantially accelerated compared to the in vitro release in simulated gastric fluid. Preferably, in this regard “substantially” means that at any given time point the in vitro release in ethanol/simulated gastric fluid (40 vol.-%) relatively deviates from the in vitro release in simulated gastric fluid by not more than +15%, more preferably not more than +10%, still more preferably not more than +8%, yet more preferably not more than +6%, even more preferably not more than +4%, most preferably not more than +2% and in particular not more than +1%.

Preferably, with the dosage forms according to the invention, a substantial relative deceleration of the in vitro release in ethanol/simulated gastric fluid (40 vol.-%) compared to the in vitro release in simulated gastric fluid is observed. In a particularly preferred embodiment, at any given time point the in vitro release in ethanol/simulated gastric fluid (40 vol.-%) relatively deviates from the in vitro release in simulated gastric fluid by at least −0.01%, more preferably at least −0.05%, still more preferably at least −0.1%, most preferably at least −0.5% and in particular at least −1%.

The dosage form according to the invention preferably exhibits resistance against solvent extraction. Preferably, the matrix provides the dosage form according to the invention with resistance against solvent extraction.

Preferably, when trying to tamper the pharmaceutical dosage form in order to prepare a formulation suitable for abuse by intravenous administration, the liquid part of the formulation that can be separated from the remainder by means of a syringe at room temperature is as little as possible, preferably it contains not more than 45 or 40 wt.-%, more preferably not more than 35 wt.-%, still more preferably not more than 30 wt.-%, yet more preferably not more than 25 wt.-%, even more preferably not more than 20 wt.-%, most preferably not more than 15 wt.-% and in particular not more than 10 wt.-% of the original content of the pharmacologically active ingredient, preferably the opioid.

Preferably, this property is tested by (i) dispensing a dosage form that is either intact or has been manually comminuted by means of two spoons in 5 ml of solvent, either purified water or aqueous ethanol (40 vol. %), (ii) allowing the dispersion to stand for 10 min at room temperature, (iii) drawing up the hot liquid into a syringe (needle 21G equipped with a cigarette filter), and (iv) determining the amount of the pharmacologically active ingredient contained in the liquid within the syringe.

The pharmaceutical dosage form according to the invention may be monolithic or multiparticulate, preferably a tablet, a capsule or a pill. Preferably, the pharmaceutical dosage form according to the invention is not in form of a film, a sheet, a membrane or in form of a matrix, a weave or a web of fibers.

Preferably, the pharmaceutical dosage form according to the invention is for use in therapy, wherein the dosage form is administered orally or perorally (upon prescribed administration, to be swallowed as a whole).

Preferably, the dosage form is not administered buccally or sublingually. Preferably, the dosage form is not adhesive to the oral mucosa.

Preferably, the pharmaceutical dosage form according to the invention is for use in therapy, wherein the dosage form is administered once daily, twice daily or thrice daily.

Further preferred embodiments of the pharmaceutical dosage form according to the invention are illustrated by the figures which, however, are not to be construed as limiting the scope of the invention.

### BRIEF DESCRIPTION OF THE DRAWINGS

FIG. 1 schematically illustrates a preferred embodiment of a pharmaceutical dosage form (1) according to the invention comprising a plurality of fibers (2) that are oriented arbitrarily, i.e. do not have a common direction of orientation.

FIG. 2 schematically illustrates another preferred embodiment of a pharmaceutical dosage form (1) according to the invention comprising a plurality of fibers (2) that with respect to layer (3) of the pharmaceutical dosage form are oriented arbitrarily, i.e. do not have a common direction of orientation.

FIG. 3 schematically illustrates another preferred embodiment of a pharmaceutical dosage form (1) according to the invention comprising a plurality of fibers (2) that with respect to layer (3) of the pharmaceutical dosage form are only aligned along the plane in x-direction and y-direction of the material. This means that essentially no fibers are aligned in the z-direction (see FIG. 3).

FIG. 4 schematically illustrates a variant of the pharmaceutical dosage form according to FIG. 3, wherein the fibers are of macroscopic size. Preferably, the fibers comprise or essentially consist of one or more polymers. The fibers are arranged essentially in parallel to one another and preferably in contact with one another thereby forming a plane which is preferably layer (3) of the pharmaceutical dosage form according to the invention (see FIG. 4).

FIGS. 5 and 6 schematically illustrate preferred embodiments of the pharmaceutical dosage form (1) according to the invention comprising layers (3a) and (3b), wherein each layer comprises fibers (2a) and (2b), respectively, which are oriented in essentially a same direction of orientation, wherein the direction of orientation of adjacent layers differs from one another (see FIGS. 5 and 6). Preferably, the angle of the two different directions of orientation of two adjacent layers is a function of the number of layers. When the pharmaceutical dosage form has n layers comprising fibers which are oriented in n different directions of orientation, the angle of the two different directions of orientation of two adjacent layers is preferably (180°/n)±10°. Thus, when the pharmaceutical dosage form has two layers (n=2), the angle of the two different directions of orientation of the two adjacent layers is preferably within the range of 90°±10°, i.e. 80° to 1000. Preferably, the direction of orientation of each layer lies essentially within the plane of said layer.

According to the embodiment of FIG. 5, the fibers (2a) and (2b) are of microscopic size and embedded in a polymer matrix.

According to the embodiment of FIG. 6, the fibers (2a) and (2b) are of macroscopic size and preferably comprise or essentially consist of one or more polymers. The fibers (2a) and (2b) are arranged essentially in parallel to one another and preferably in contact with one another thereby forming planes which are preferably layers (3a) and (3b) of the pharmaceutical dosage form according to the invention

FIG. 7 schematically illustrates another preferred embodiment of the pharmaceutical dosage form (1) according to the invention comprising wherein the fibers (2) in form a woven or nonwoven fabric (4) surrounding an inner core which comprises the pharmacologically active ingredient. The dosage form comprises an outer coating (5) of excipients deposited at the outer surface of the fabric (4) such that it is not visible from the outside.

FIG. 8 schematically illustrates a variant of the pharmaceutical dosage form according to FIG. 7, wherein the fabric surrounds the core of the pharmaceutical dosage form in a pouf-like arrangement.

FIG. 9 schematically illustrates a preferred embodiment of a pharmaceutical dosage form (1) according to the invention comprising pockets (6) that—once their ends are exposed to gastric fluids—serve as canals allowing the release medium, e.g. the gastric fluid, to penetrate from the outside through the pockets (6) into the pharmaceutical dosage form i.e. into its interior and inner core, respectively.

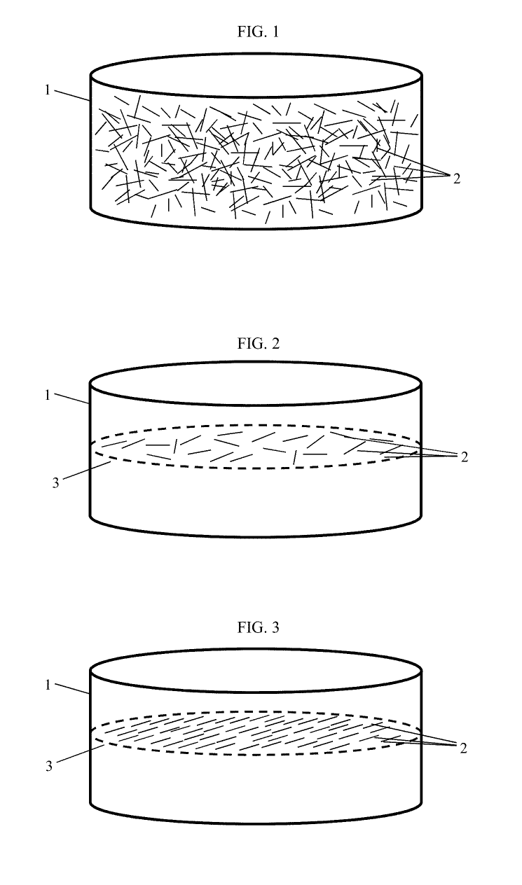

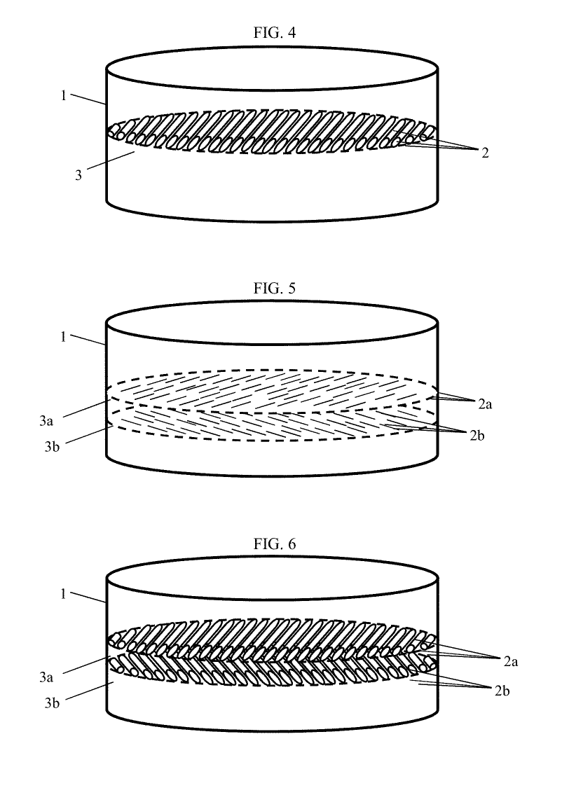

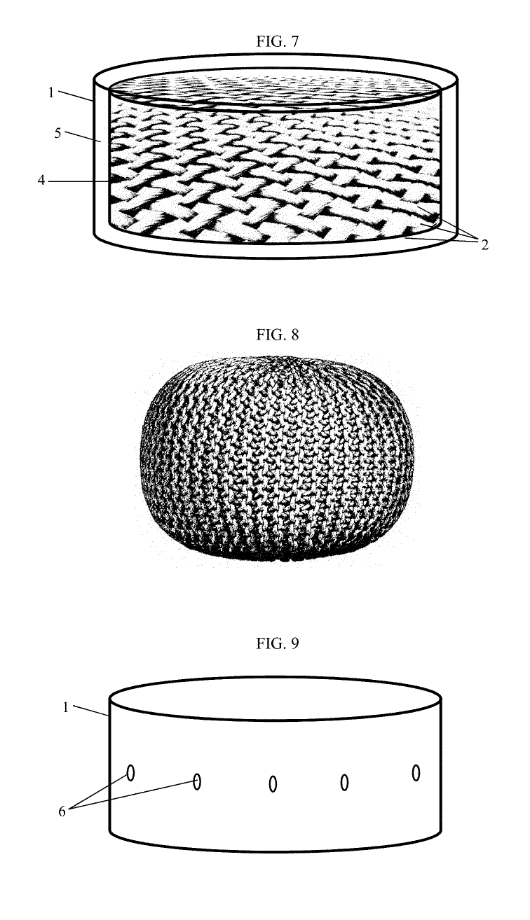

The pharmaceutical dosage form according to the invention can be manufactured by conventional means, such as direct compression, granulation (dry or wet) or extrusion.

In a preferred embodiment, the pharmaceutical dosage form according to the invention is thermoformed, e.g. hot-melt extruded.

The pharmaceutical dosage form according to the invention is preferably produced by mixing the pharmacologically active ingredient, the fibers and all additional excipients and, optionally after granulation, press-forming the resultant mixture to yield the dosage form with preceding, simultaneous, or subsequent exposure to heat.

A powder mixture may be heated and then subsequently compressed, or it may be heated and simultaneously compressed, or it may be compressed and then subsequently heated.

Mixing proceeds in a mixer known to the person skilled in the art. The mixer may, for example, be a roll mixer, shaking mixer, shear mixer or compulsory mixer.

The resultant mixture is preferably formed directly by application of pressure to yield the dosage form according to the invention with preceding, simultaneous or subsequent exposure to heat. The mixture may, for example, be formed into tablets by direct tabletting. In direct tabletting with simultaneous exposure to heat, the tabletting tool, i.e. bottom punch, top punch and die are briefly heated at least to the softening temperature of the polymers that are contained in the polymer matrix and pressed together. In direct tabletting with subsequent exposure to heat, the formed tablets are briefly heated at least to the softening temperature (glass transition temperature, melting temperature; sintering temperature) of the polymers and cooled again. In direct tabletting with preceding exposure to heat, the material to be pressed is heated immediately prior to tabletting at least to the softening temperature of the polymers and then pressed.

The resultant mixture may also first be granulated and then be formed with preceding, simultaneous, or subsequent exposure to heat to yield the dosage form according to the invention.

Another aspect of the invention relates to a process for the preparation of a dosage form according to the invention as described above, said process comprising a three-dimensional printing step. It has been surprisingly found that pharmaceutical dosage forms comprising comparatively large cavities can be manufactured by three-dimensional printing technologies.

Preferably, the three-dimensional printing step involves fused deposition modeling.

Machines for fused deposition modeling (FDM) are commercially available. The machines may dispense multiple materials to achieve different goals: For example, one material may be used to build up the pharmaceutical dosage form and another material may be used to build up a soluble support structure.

In FDM the pharmaceutical dosage form is produced by extruding small flattened strings of molten material to form layers as the material hardens immediately after extrusion from the nozzle. A thermoplastic filament is unwound from a coil and supplies material to an extrusion nozzle which can turn the flow on and off. A worm-drive may push the filament into the nozzle at a controlled rate. The nozzle is heated to melt the material. The thermoplastic material is heated above its glass transition temperature and is then deposited by an extrusion die. The nozzle can be moved in both horizontal and vertical directions by a numerically controlled mechanism. The nozzle follows a tool-path controlled by a computer-aided manufacturing (CAM) software package, and the pharmaceutical dosage form is built from the bottom up, one layer at a time. Stepper motors or servo motors are typically employed to move the extrusion die. The mechanism used is often an X—Y—Z rectilinear design, although other mechanical designs such as deltabot have been employed. Myriad materials are commercially available, such as polylactic acid (PLA), polyamide (PA), among many others (see Ursan et al., J Am Pharm Assoc (2003) 2013, 53(2), 136.44; Prasad et al., Drug Dev Ind Pharm 2015, 1-13).

Pharmaceutical compositions that are suitable to be employed in the three-dimensional printing step according to the invention, preferably in fused deposition modeling, are preferably identical to or at least similar with pharmaceutical compositions that have been known to be suitable for processing by conventional hot melt extrusion technology. Fused deposition modeling has many similarities with conventional hot melt extrusion.

A representative pharmaceutical composition is summarized in the table here below:

| Constituent | mg | wt.-% |
|---|---|---|
| Tramadol | 100 | 40 |
| PEG 4000 | 30 | 12 |
| PEEK | 100 | 40 |
| HPMC | 20 | 8 |

The pharmacologically active ingredient (here Tramadol) is mixed with the cut-resistant thermoplastic material in an extruder thereby providing a three-dimensionally printable filament having a diameter within the range of e.g. from 1.0 to 5.0 mm.

Preferably, the pharmaceutical dosage form is prepared by three-dimensionally printing at least two different pharmaceutical compositions that preferably are provided each in form of filaments useful for fused deposition modeling. Preferably, one pharmaceutical composition contains one or more pharmacologically active ingredients, whereas the other pharmaceutical composition does not contain pharmacologically active ingredients.

Both compositions preferably contain pharmaceutical excipients that are conventionally employed in the manufacture of pharmaceutical dosage forms, preferably in the course of three-dimensional printing technology, especially fused deposition modeling. The following preferred embodiments apply to both pharmaceutical compositions (in the following referred to as “pharmaceutical composition”), irrespective of whether they contain a pharmacologically active ingredient or not.

Preferably, the pharmaceutical composition comprises a plasticizer. Suitable plasticizers are known to the skilled person. Examples include but are not limited to polyethylene glycols, such as PEG 1500 or PEG 4000 or PEG 6000; citrates, phthalates, glycerin, sugar alcohols, various contents of copolymers (e.g. ethylene vinyl acetate (EVA)/vinyl acetate (VA)), and mixtures of any of the foregoing.

The content of plasticizer is preferably within the range of from 0.1 to 20 wt.-%, more preferably 5.0 to 17.5 wt.-%, still more preferably 7.5 to 15 wt.-%, relative to the total weight of the pharmaceutical composition.

For filament preparation, a matrix polymer or a mixture of various matrix polymers, e.g. hydroxypropylcellulose (HPC), may be stored 24 h in oven at 40° C.; when required it may be mixed in a mortar with PEG 1500 or PEG 4000 (2%, 5%, 10% by weight calculated with respect to the dry polymer). Hot-melt extrusion (HME) may be carried out in a twin-screw extruder (Haake MiniLab II, Thermo Scientific, USA) equipped with an aluminum rod-shaped die (ø2.00 mm). Extruded rods may be calibrated and rolled up on a spool.

Another aspect of the invention relates to a pharmaceutical dosage form that is obtainable by the process according to the invention as described above.

1.-87. (canceled)

88. A reinforced pharmaceutical dosage form comprising a pharmacologically active ingredient and fibers; wherein the pharmaceutical dosage form comprises a polymer matrix that is reinforced with the fibers; wherein the polymer matrix comprises a polymer selected from polyether ether ketones; and wherein the pharmaceutical dosage form is a tablet, a capsule or a pill.

89. The pharmaceutical dosage form according to claim 88, wherein the fibers are manufactured by three-dimensional printing technology.

90. The pharmaceutical dosage form according to claim 88, wherein at least a portion of the fibers is oriented in essentially the same direction.

91. The pharmaceutical dosage form according to claim 88, which comprises a plurality of layers, wherein each layer comprises fibers which are oriented in essentially a same direction of orientation, wherein the direction of orientation of adjacent layers differs from one another.

92. The pharmaceutical dosage form according to claim 91, wherein the direction of orientation of all layers differs from one another.

93. The pharmaceutical dosage form according to claim 91, wherein the direction of orientation of each layer lies essentially within the plane of said layer.

94. The pharmaceutical dosage form according to claim 88, wherein the polymer matrix is manufactured by three-dimensional printing technology.

95. The pharmaceutical dosage form according to claim 88, wherein the fibers are selected from the group consisting of glass fibers, carbon fibers, mineral fibers, polymer fibers, and mixtures thereof.

96. The pharmaceutical dosage form according to claim 95, wherein the fibers are polymer fibers comprising a polymer selected from the group consisting of polyesters, polyamides, polyurethanes, cellulose ethers, polyacrylates, vinyl polymers, polyether ether ketones, polyalkylene oxides, and mixtures thereof.

97. The pharmaceutical dosage form according to claim 88, wherein the pharmacologically active ingredient has psychotropic action.

98. The pharmaceutical dosage form according to claim 88, wherein the pharmacologically active ingredient is selected from opioids and stimulants.

99. The pharmaceutical dosage form according to claim 88, which is tamper resistant.

100. The pharmaceutical dosage form according to claim 88, which comprises one or more pockets that serve as canals allowing the release medium to penetrate from the outside through the pockets into the pharmaceutical dosage form.

101. A method for treating a condition in a patient in need thereof, said method comprising orally administering to said patient the pharmaceutical dosage form according to claim 88, wherein the pharmacologically active ingredient is effective for said treating.

102. A process for preparing a dosage form according to claim 88, said process comprising a three-dimensional printing step.
